(19) United States
(12) Patent Application Publication
(10) Pub. No.: US 2010/0316566 A1
(43) Pub. Date: Dec. 16, 2010

(54) RADIOLABELED HEDGEHOG DERIVATIVES FOR IMAGING AND THERAPY

(75) Inventors: Jennifer Sims-Mourtada, Bellaire, TX (US); Ali Azhdarinia, Houston, TX (US); Izabela Tworowska, Houston, TX (US); Hitomi Saso, Houston, TX (US)

Correspondence Address: OSHA LIANG L.L.P., TWO HOUSTON CENTER, 909 FANNIN, SUITE 3500, HOUSTON, TX, 77010, US

(73) Assignee: RADIOMEDIX INC., Houston, TX (US)

(21) Appl. No.: 12/918,443

(22) PCT Filed: Feb. 27, 2009
(86) PCT No.: PCT/US09/35489

§ 371 Date: Aug. 19, 2010

Related U.S. Application Data
(60) Provisional application No. 61/032,054, filed on Feb. 27, 2008.

(51) Int. Cl. A61K 51/00; A61K 49/00; A61K 33/24; A61K 33/36; A61K 33/26; A61P 35/04

(52) U.S. Cl. 424/1.65; 424/9.1; 424/617; 424/629; 424/630; 424/646; 424/649; 424/650

(57) ABSTRACT

The present invention concerns methods and compositions related to a chelator and a HHRT ligand. In specific embodiments of the invention the chelator is conjugated to the HHRT ligand. In another specific embodiment of the invention, the chelator is chelated to a metal. In a particular embodiment of the invention, there is a metal species that is chelated to a chelator, which is then directly or indirectly conjugated to a HHRT ligand. In some embodiments, the composition further comprises a therapeutic agent. In particular cases, the compositions are employed for cancer diagnosis and/or therapy.

### FIELD OF THE INVENTION

This invention describes novel compounds comprising a Hedgehog receptor targeting ligand, a chelator and a metal. The invention describes methods for diagnosing, monitoring and/or treating cancer. In particular, this invention relates to diagnosis, monitoring and/or treatment of hedgehog receptor PATCHED-expressing tumors with targeted radiopharmaceuticals. The present invention is related at least to the fields of radiochemistry, nuclear imaging, radionuclide therapy, cell biology, molecular biology, medicine, and chemical synthesis.

### BACKGROUND OF THE INVENTION

The hedgehog (HH) signaling pathway is critical for growth and differentiation during embryonic development (Ingham and McMahon 2001). Secreted HH molecules (Sonic, Desert and Indian) bind to and inhibit the cell surface receptor PATCHED (PTCH). This binding relieves the PTCH-mediated suppression of the transmembrane protein SMOOTHENED (SMO) leading to multiple intracellular events that result in the nuclear translocation and activation of the Gli family of transcription factors (Gli-1, 2 and 3). (Ingham and McMahon 2001; Ruel, Rodriguez et al. 2003) Transcriptional targets of Gli-1 include genes controlling cell cycle, cell adhesion, signal transduction, vascularization and apoptosis. (Yoon, Kita et al. 2002) Additionally, Gli-1 regulates transcription of both PTCH and itself (Dai, Akimaru et al. 1999)

Overexpression of the HH signaling pathway has been identified in many cancers, including basal cell carcinoma (Couve-Privat, Le Bret et al. 2004), medulloblastoma (Rao, Pedone et al. 2004), hepatocellular carcinoma (Osipo and Miele 2006; Patil, Zhang et al. 2006; Sicklick, Li et al. 2006), pituitary carcinoma (Watkins, Berman et al. 2003; Vila, Theodoropoulou et al. 2005), glioblastoma (Ehtesham, Sarangi et al. 2007) (Bar, Chaudhry et al. 2007), cartilaginous tumors (Park and Park 2007), breast cancer (Mukherjee, Frolova et al. 2006), prostate cancer (Sheng, Li et al. 2004; Anton Aparicio, Garcia Campelo et al. 2007), uterine and cervical cancer (Xuan, Jung et al. 2006), ovarian cancer (Levanat, Musani et al. 2004; Steg, Wang et al. 2006), small cell lung cancer (Vestergaard, Pedersen et al. 2006), urothelial carcinoma (Thievessen, Wolter et al. 2005), squamous cell carcinoma (Snijders, Schmidt et al. 2005; Xuan, Jung et al. 2006), gastric cancer (Ma, Chen et al. 2005; Fukaya, Isohata et al. 2006; Lee, Han et al. 2007), esophageal cancer (Ma, Sheng et al. 2006; Sui, Bonde et al. 2006), pancreatic cancer (Liu, Yang et al. 2007; Morton, Mongeau et al. 2007), kidney cancer (Cutcliffe, Kersey et al. 2005), multiple myeloma (Peacock, Wang et al. 2007) and leukemia (Sengupta, Banerjee et al. 2007).

The association between the HH pathway and cancer was initially established by the identification of heterozygous mutations affecting the membrane receptor PTCH, resulting in abnormal activation of HH signaling in basal cell carcinoma and neural tumors. (Bale and Yu 2001; Harmon, Ko et al. 2002) Recently, several studies have shown constitutive, ligand-dependent activation of the HH signaling pathway in multiple cancers, suggesting that unregulated progenitor cell proliferation induced by abnormal HH signaling has a role in carcinogenesis. (Bale and Yu 2001; Harmon, Ko et al. 2002; Berman, Karhadkar et al. 2003; Thayer, di Magliano et al. 2003; Watkins and Peacock 2004; Ma, Sheng et al. 2005)

Studies have shown that HH signaling contributes to radiation and chemotherapeutic resistance in tumors through regulation of survival proteins, cell cycle, DNA repair and drug transport. (Shafaee, Schmidt et al. 2006; Sims-Mourtada, Izzo et al. 2006; Sims-Mourtada, Izzo et al. 2007) In addition to cancer, abnormal HH signaling has been implicated in other disorders including chronic inflammation of the gastric mucosa, esophagus (Dimmler, Brabletz et al. 2003; Nielsen, Williams et al. 2004) (Kayed, Kleeff et al. 2005) and inflammatory liver injury induced by ischemia/reperfusion. (Tuncer, Ozturk et al. 2007)

Detection of hedgehog signaling in tumors is currently possible in surgical samples or biopsies using immunohistochemistry or quantitative PCR. However, non-invasive detection of PTCH expression with diagnostic imaging techniques provides advantages over traditional methods, including real time monitoring and elimination of biopsy sampling bias.

Radiolabeled receptor binding peptides and proteins have emerged as an important class of radiopharmaceuticals for functional imaging and targeted treatment of cancer. Specific receptor binding properties of ligands can be exploited by labeling the protein or peptide with a radionuclide. The radiolabeled ligand can then be used as a vehicle to deliver radioactivity to the tissues expressing a particular receptor, such as hedgehog receptor targeting (HHRT) ligands.

Receptor binding peptides and proteins have been radiolabeled with gamma emitters such as ¹²³I, ¹¹¹In and ^{99m}Tc for SPECT imaging and ¹⁸F, ¹⁵O, ¹¹C, ⁶⁸Ga, ⁶⁴Cu and ¹²⁴I for PET imaging. For targeted radiotherapy, receptor binding peptides and proteins can be labeled with cytotoxic, β-emitting radionuclide like ¹³¹I and ¹⁷⁷Lu.

Improvement of scintigraphic tumor diagnosis, prognosis, planning, and monitoring of treatment of cancer is intimately linked with the development of more tumor-specific radiopharmaceuticals, such as hedgehog receptor targeting (HHRT) ligands. As a result, molecular nuclear medicine is improving methodologies for tumor diagnosis and staging, the monitoring of tumor response to treatment, and prediction of therapeutic response through the development and characterization of novel radiotracers.

Similarly, therapeutic nuclear medicine has benefited from the discovery and validation of novel molecular targets. Identifying specific molecules associated with certain diseases has lead to the development of targeted biomolecules that carry a therapeutic radionuclide as a payload. This results in specific delivery of radioactivity to the desired site while sparing non-target organs from unnecessary radiation dose.

### SUMMARY OF THE INVENTION

The present invention is directed to compositions and methods for a radiopharmaceutical targeting a selected biological site. More particularly, it employs radiolabeling PTCH targeting (i.e. HHRT) ligands, for example, for methods of using those radiolabeled hedgehog ligands for imaging, and/or radionuclide therapy, including tissue-specific disease imaging and/or therapy.

The present invention overcomes limitations in regards to the lack of targeted radionuclide cancer therapy and other drawbacks of the prior art by providing a new radiolabeling strategy to target PTCH receptor positive tumors for imaging, diagnosis, and treatment. The invention provides versatile HH-like drug conjugates which can be labeled with various radioactive and non-radioactive metals, as well as methods for making the radiolabeled ligands and for using them to image and treat cancer.

Among the advantages found to be achieved by the present invention include the ability for diagnosing and staging of tumors. For example, Sheng et al. reported high levels of PTCH expression in over 70% of prostate tumors with Gleason scores 8-10, but only in 22% of tumors with Gleason less than 6, indicating that PTCH receptor expression correlates with tumor aggressiveness (Sheng, Li et al. 2004). In addition, high levels of PTCH expression were reported in 100% of prostate cancer metastases examined, a finding which has been supported by subsequent studies (Karhadkar, Bova et al. 2004; Sanchez, Hernandez et al. 2004). Thus, radiolabeling HH ligands that bind to PTCH with ⁶⁸Ga or ^{99m}Tc can provide for staging of prostate cancer by PET or SPECT, respectively.

Similarly, in a study to identify biomarkers associated with resistant tumors, the phenomenon of upregulation of the HH pathway in residual esophageal adenocarcinoma specimens from patients who failed to respond to pre-operative chemotherapy and radiation (Sims-Mourtada et al., 2006; Yoshikawa et al, 2008). Monitoring activity of this pathway allows for prediction and early monitoring of treatment responses, in particular embodiments. In this example, ⁶⁸Ga-DOTA-SHH provides a method to monitor treatment responses by PETduring the early stages of therapy.

The present invention also provides a method to treat tumors by targeting high dose radiation to tumor cells. Because PTCH is overexpressed in androgen independent and advanced prostate tumors, radiolabeled HH ligands can provide a novel approach for the specific delivery of high-dose radiation directly to the tumor cells, with limited systemic toxicity. Moreover, HH targeted radionuclide therapy may effectively target tumor progenitor cells which are implicated in disease reoccurrence following treatment with traditional cancer therapies and are often found in highly aggressive or metastatic tumors. In this example, HH ligands radiolabeled with the therapeutic radionuclide ¹⁷⁷Lu provides a method for targeting the radioactive payload directly to PTCH positive tumor cells.

In some embodiments of the invention, the cancer to be diagnosed and/or treated is cancer that is resistant to one or more therapies, including resistant to hormone treatment, for example. In particular embodiments, the cancer cells to be treated overexpress the hedgehog receptor PTCH on the surface of the cell. The cancer may be of any kind of cancer, including a solid tumor or a cancer that is not a solid tumor. In cases wherein the cancer is breast cancer, for example, it may be estrogen receptor (ER) positive or negative, or progesterone receptor (PR) positive or negative. The breast cancer may be Her2/neu positive or negative. In some cases, the cancer is androgen receptor positive or negative. In specific examples, the cancer cells to be targeted with the methods and compositions of the present invention are cancer stem cells.

In some cases wherein a medical condition is diagnosed, the individual is provided a composition of the present invention, wherein the presence of the composition upon imaging identifies a particular medical condition. In other cases, the absence of the composition upon imaging identifies a particular medical condition. In specific examples, one may remove stem cells from the bone marrow of the individual and replace them with stem cells. These replaced stem cells are then targeted by a composition of the present invention, and the presence of such compositions upon imaging of the bone marrow identifies the stem cells as proliferating within the bone marrow of the individual.

In specific embodiments, the cancer is a solid tumor, and it may be imaged or treated with compositions of the present invention. In cases wherein the cancer is not in a solid tumor, for example, leukemia, it may be treated with a composition of the present invention. Diagnosis of a non-solid tumor may be useful only within a particular region, such as bone marrow, for example.

The general embodiment of the invention concerns a chelator and a HHRT ligand. In specific embodiments of the invention the chelator is conjugated to the HHRT ligand. In a certain embodiment of the invention, there is a metal species that is chelated to a chelator, which is then directly or indirectly conjugated to a HHRT ligand.

In another embodiment of the invention, the HHRT is any molecule that binds to PTCH. In specific embodiments, the HHRT is a small molecule or anti-cancer drug, for example. In a specific embodiment, the HHRT is a HH peptide. In another embodiment the HH peptide is further defined as a polypeptide of 10 or more amino acids with at least 70% homology to the native HH ligand. SEQ ID NO:11 (GenBank® Accession NO.: NP_{—}066382; SEQ ID NO:12 (GenBank® Accession NO:NP_{—}002172).

In another embodiment of the invention, the chelator is comprised of a combination of N, O, and S atoms. In a specific embodiment, the chelator is a tetraaza compound. In another embodiment of the invention, the chelator is further defined as a transition chelator. This chelator could be of the group of glucoheptanate, gluconate, glycarate, citrate, tartarate, DOTA, diethylenetriaminepentaacetic acid or ethylenediaminetetraacetic acid.

In a general embodiment, the invention is a therapeutic and/or diagnostic composition. Another general embodiment is the method of treating a subject for a medical condition by administering to the subject a composition of the instant invention. In another embodiment, the instant invention is used in a method of diagnosing a subject for a medical condition. In a specific embodiment of the invention, the subject is a mammal, for example a human, dog, cat, horse, goat, sheep, or pig. In a further embodiment, the invention is administered concurrently, subsequently, or prior to an additional cancer therapy and/or diagnosis means, such as another form of radiation therapy or surgery, for example. In one embodiment, the medical condition is cancer.

In a specific embodiment, the compositions and methods of the invention concern targeting cells that overexpress PTCH, including cancer cells that overexpress PTCH.

In certain embodiments, the site targeted by compositions of the invention will be a tumor, heart, lung, brain, liver, spleen, pancreas, intestine or any other organ. The tumor may be located anywhere within the mammalian body but in some embodiments is in the breast, ovary, prostate, endometrium, lung, brain, pancreas, or liver, for example.

In one embodiment of the invention, a composition of the invention comprises a pharmaceutically acceptable excipient or a carrier.

In another embodiment, the instant invention is utilized for imaging, including for diagnostic imaging, for example. In a specific embodiment, the imaging comprises PET or SPECT imaging.

In certain cases, the composition of the invention is comprised in a kit. In a further embodiment, the kit also comprises an oxidizing agent. In another embodiment, the kit also comprises a reducing agent in cases where isotopes such as ^{99m}Tc or ¹⁸⁶/¹⁸⁸Re are used for radiolabeling.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

In the field of nuclear medicine, certain pathological conditions are localized, or their extent is assessed, by detecting the distribution of small quantities of internally-administered radioactively labeled tracer compounds (which may be referred to as radiotracers or radiopharmaceuticals). Methods for detecting these radiopharmaceuticals are known generally as “imaging” or “radioimaging” methods, for example.

The term “derivative” as used herein is a compound that is formed from a similar compound or a compound that can be considered to arise from another compound, if one atom is replaced with another atom or group of atoms. Derivative can also refer to compounds that at least theoretically can be formed from the precursor compound.

As used herein, the word “compound” means a free chemical molecular entity or a chemical moiety that is part of a larger molecular entity. Therefore, when reference is made, for example, to a targeting ligand being an anti-cancer compound, the language encompasses both an anti-cancer compound moiety incorporated within a larger chemical entity as well as the free anticancer compound.

The word “conjugate” and “conjugated” is defined herein as chemically joining within the same molecule. For example, two or more molecules and/or atoms may be conjugated together via a covalent bond, forming a single molecule. The two molecules may be conjugated to each other via a direct connection (e.g., where the compounds are directly attached via a covalent bond) or the compounds may be conjugated via an indirect connection (e.g., where the two compounds are covalently bonded to one or more linkers, forming a single molecule). In other instances, a metal atom may be conjugated to a molecule via a chelation interaction.

As used herein the term “radionuclide” is defined as a radioactive nuclide (a species of atom able to exist for a measurable lifetime and distinguished by its charge, mass, number, and quantum state of the nucleus) which, in specific embodiments, disintegrates with emission of corpuscular or electromagnetic radiation. The term may be used interchangeably with the term “radioisotope”.

The term “therapeutic agent” as used herein is defined as an agent which provides treatment for a disease or medical condition. The agent in a specific embodiment improves at least one symptom or parameter of the disease or medical condition. For instance, in tumor therapy, the therapeutic agent reduces the size of the tumor, inhibits or prevents growth or metastases of the tumor, or eliminates the tumor. Examples include a drug, such as an anticancer drug, a gene therapy composition, a radionuclide, a hormone, a nutriceutical, or a combination thereof.

The term “tumor” as used herein is defined as an uncontrolled and progressive growth of cells in a tissue. A skilled artisan is aware other synonymous terms exist, such as neoplasm or malignancy. In a specific embodiment, the tumor is a solid tumor. In other specific embodiments, the tumor derives, either primarily or as a metastatic form, from cancers such as of the liver, prostate, pancreas, head and neck, breast, brain, colon, adenoid, oral, skin, lung, testes, ovaries, cervix, endometrium, bladder, stomach, and epithelium.

The term “drug” as used herein is defined as a compound which aids in the treatment of disease or medical condition or which controls or improves any physiological or pathological condition associated with the disease or medical condition.

The term “anti-cancer compound” as used herein is defined as a drug for the treatment of cancer, such as for a solid tumor. The anticancer drug preferably reduces the size of the tumor, inhibits or prevents growth or metastases of the tumor, and/or eliminates the tumor. The terms “anticancer drug”, “anti-cancer drug”, and “anti-cancer compound” are used interchangeably herein.

The term “chelator” as used herein is used to describe complexes in which a metal ion could be bound to two or more atoms of the chelator, in which the bonds may be any combination of coordination or ionic bonds.

The term “pharmaceutically acceptable excipient” as used herein is intended to include any substance capable of being admixed and administered with the instant invention and which allows the invention to perform its intended function as disclosed herein. Pharmaceutically acceptable excipient includes any physiologically inert, pharmacologically inactive material known to one skilled in the art, which is compatible with the physical and chemical characteristics of the particular active ingredient selected for use. Excipients suitable for use include, but are not limited to, proteins such as gelatin, polymers, resins, plasticizers, fillers, binders, lubricants, glidants, disintegrates, solvents, co-solvents, buffer systems, surfactants, preservatives, sweetening agents, flavoring agents, pharmaceutical grade dyes or pigments, and viscosity agents. It is within the skill of the ordinary practitioner using no more than routine experimentation to identify a suitable excipient.

The term “transition chelator” as used herein is any chelator molecule that can chelate any transition metal. Transition chelators need not be chelated to a transition metal, but are only required to have the possibility of being chelated to a transition metal. Transition chelators may also be able to chelate other categories of metals,

The term “antioxidant” as used herein is a molecule capable of slowing or preventing the oxidation of other molecules, wherein oxidation refers to the loss of one or more electrons.

The term “reducing agent” as used herein refers to a molecule that donates electors, thereby reducing other molecules while being oxidized itself.

The term “delivering” as used herein is defined as brining to a destination and includes administering, as for a therapeutic purpose.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, horses, cows, goats, sheep, mice, rats, and chimpanzees.

The term “treatment” refers to any process, action, application, therapy, or the like, wherein a mammal, including a human being, is subject to medical aid with the object of improving the mammal's condition, directly or indirectly. In some embodiments, one or more symptoms of the mammal's condition are alleviated at least partially.

The term “therapeutically effective” as used herein is defined as the amount of a compound required to improve a disease. For example, in the treatment of cancer, a compound which reduces proliferation of the cells, reduces tumor size, reduces metastases, reduces proliferation of blood vessels to said cancer, facilitates an immune response against the cancer would be therapeutically effective. A therapeutically effective amount of a compound is not required to cure a disease but will provide a treatment for a disease.

### II. Hedgehog Receptor Targeting Ligands

The HH receptor targeting ligand may be of any suitable kind. “Hedgehog receptor targeting” or “HHRT” refers to the ability of a compound to preferentially associate with PTCH receptor positive cells (e.g., cancerous, pre-cancerous, and/or benign). A “hedgehog receptor targeting ligand” refers to a compound that preferentially binds to or associates with the PTCH receptor. The ligand may be, but is not limited to, a small molecule, drug, peptide, or protein, for example. “Targeting ligand” or “targeting moiety” may be used in the same context interchangeably.

A. Hedgehog Protein

The HH signaling pathway is one of the key regulators of animal development conserved across species. As stated above, HH signaling is overrepresented in certain types of cancers. Mammals have three HH homologues; Sonic, Indian, and Desert. All three can bind to PTCH receptors with similar binding affinities.

In certain cases, human SHH is provided as SEQ ID NO:10 (CGPGRG FGKRRHPKKL TPLAYKQFIP NVAEKTLGAS GRYEGKITRN SERFKELTPN YNPDIIFKDE ENTGADRLMT QRCKDKLNAL AISVMNQWPG VKLRVTEGWD EDGHHSEESL HYEGRAVDIT TSDRDRSKYG MLARLAVEAG FDWVYYESKA HIHCSVKAEN SVAAKSG).

In certain cases, human DHH is provided as SEQ ID NO 11: (MALLTNLLPL CCLALLALPA QSCGPGRGPV GRRRYARKQL VPLLYKQFVP GVPERTLGAS GPAEGRVARG SERFRDLVPN YNPDIIFKDE ENSGADRLMT ERCKERVNAL AIAVMNMWPG VRLRVTEGWD EDGHHAQDSL HYEGRALDIT TSDRDRNKYG LLARLAVEAG FDWVYYESRN HVHVSVKADN SLAVRAGGCF PGNATVRLWS GERKGLRELH RGDWVLAADA SGRVVPTPVL1 LFLDRDLQRR ASFVAVETEW PPRKLLLTPW HLVFAARGPA PAPGDFAPVF ARRLRAGDSV LAPGGDALRP ARVARVAREE AVGVFAPLTA HGTLLVNDVL ASCYAVLESH QWAHRAFAP RLLHALGALL PGGAVQPTGM HWYSRLLYRL AEELLG)

In certain cases, human IHH is provided as SEQ ID NO 12: (MSPARLRPRL HFCLVLLLLL VVPAAWGCGP GRVVGSRRRP PRKLVPLAYK QFSPNVPEK TLGASGRYEGK IARSSERFKE LTPNYNPDII FKDEENTGAD RLMTQRCKDR LNSLAISVMN QWPGVKLRVT EGWDEDGHHS EESLHYEGRA VDITTSDRDR NKYGLLARLA VEAGFDWVYY ESKAHVHCSV KSEHSAAAKT GGCFPAGAQV RLESGARVAL SAVRPGDRVL AMGEDGSPTF SDVLIFLDRE PHRLRAFQVI ETQDPPRRLA LTPAHLLFTA DNHTEPAARF RATFASHVQP GQYVLVAGVP GLQPARVAAV STHVALGAYA PLTKHGTLVV EDVVASCFAA VADHHLAQLA FWPLRLFHSL AWGSWTPGEG VHWYPQLLYR LGRLLLEEGS FHPLGMSGAG S)

B. Hedgehog Protein Derivatives

In certain cases, derivatives of HH are employed, including those that are identical to SEQ ID NO:10, or those that are comprised within SEQ ID NO:10, some of which may or may not have alterations compared to the corresponding sequence in SEQ ID NO:10. In specific embodiments, the derivative is at least 172 amino acids in length, at least 170 amino acids in length, at least 165 amino acids in length, at least 160 amino acids in length, at least 155 amino acids in length, at least 150 amino acids in length, at least 145 amino acids in length, at least 140 amino acids in length, at least 135 amino acids in length, at least 130 amino acids in length, at least 125 amino acids in length, at least 120 amino acids in length, at least 115 amino acids in length, at least 110 amino acids in length, at least 105 amino acids in length, at least 100 amino acids in length, at least 90 amino acids in length, at least 80 amino acids in length, at least 70 amino acids in length, at least 60 amino acids in length, at least 50 amino acids in length, at least 40 amino acids in length, at least 30 amino acids in length, at least 20 amino acids in length, or at least 10 amino acids in length. In specific embodiments, the derivative is 70% or more identical to SEQ ID NO:10, 75% or more identical to SEQ ID NO:10, 80% or more identical to SEQ ID NO:10, 85% or more identical to SEQ ID NO:10, 90% or more identical to SEQ ID NO:10, 95% or more identical to SEQ ID NO:10, 97% or more identical to SEQ ID NO:10, or 99% or more identical to SEQ ID NO:10.

### III. Chelators

The present invention provides a method by which bifunctional chelators, in certain embodiments, are conjugated to HHRT ligands to produce novel compounds that may be used for purposes including imaging, diagnosis, treatment, and/or radiotherapy.

A. Bifunctional Chelators

Chelators that bind radionuclides and are conjugated to biomolecules are referred to as bifunctional chelating agents (BFCAs). The use of various BFCAs for radiolabeling molecules is well known in the art. BFCAs serve two main purposes: 1) to coordinate the radiometal; and 2) to provide a molecular backbone that can be modified with functional groups for attachment to the targeting biomolecule. The BFCA is conjugated to the molecule of interest in a manner that does not interfere or adversely affect the binding properties or specificity of the molecule.

Suitable BFCAs are generally multidentate (typically at least tetradentate) and are comprised of electron-rich atoms such as nitrogen, oxygen, sulfur and phosphorus. Chelates for inclusion in the present application are selected based on the metal to be incorporated and the clinical objectives. Chelates selected for use in the present invention include, but are not limited to, those listed below:
HYNIC, DMSA, N₂S₂ chelators, MAG3, EDTA, DTPA, cyclen, bridged-cyclam, et-cyclam, cylamdione, DOTA, TRITA, TETA, bridged-cyclam-2a, DO3A, DO2A, DO2S, NOTA, DOTP, DO3P and DO2P.

B. Transition Chelators

In some embodiments of the invention, a transition chelator is employed. Although any transition chelator may be employed, in specific embodiments, it is glucoheptanate, glyconate, glycarate, citrate, tartarate, DOTA, diethylenetriaminepentaacetic acid or ethylenediaminetetraacetic acid.

### IV. Formulations of Chelator Derivatives

To quench the bioconjugation reaction, a transchelator can be added to the radiotracer to remove any free radioisotope. Examples of acceptable transchelators for radionuclides include polycarboxylic acids, e.g., tartrate, citrate, phthalate, iminodiacetate, DOTA, ethylenediaminetetraacetic acid (EDTA), diethylenetriaminepentaacetic acid (DTPA) and the like. Additionally, any of a variety of anionic and/or hydroxylic oxygen-containing species could serve this function, e.g., salicylates, acetylacetonates, hydroxyacids, catechols, glycols and other polyols, e.g., glucoheptonate, and the like. Other suitable reagents and protocols for the formulation of radiopharmaceuticals will be apparent to those skilled in the art and may be readily adapted for use with the apparatus of the present invention.

### V. Conjugates

The term “BFCA-HHRT ligand conjugate” is defined herein as a HHRT ligand that has been conjugated to a BFCA. In certain embodiments the BFCA-HHRT ligand conjugate comprises a chelator that has at least one atom chelated to it. The BFCA-HHRT ligand conjugate may comprise a BFCA that is conjugated to a targeting ligand (e.g., via a covalent bond) and/or a metal chelate (e.g., via a chelation interaction).

In certain aspects, the derivatives have a metal atom chelated to them (i.e., the conjugate may be labeled with a radioisotope). The metal atom may be radioactive or non-radioactive, in particular cases.

Yet another embodiment of the present invention is a reagent for preparing a scintigraphic imaging agent. The reagent of the invention includes a HHRT ligand, having an affinity for targeted sites in vivo sufficient to produce a scintigraphically-detectable image, covalently linked to a radiolabeled BFCA moiety. The radiolabeled BFCA moiety is directly attached to the HHRT ligand. For ⁶⁸Ga, the binding moiety is preferably a macrocyclic chelate containing a tri-aza or tetraza core. For example, the HHRT ligand may be covalently linked to a carboxygroup of DOTA. The HHRT ligand may be any of the ligands as described above.

Conjugation of BFCAs can be applied to multiple classes of HHRT ligands described herein. In certain embodiments, these bioconjugates could then be radiolabeled using the apparatus of the present invention through an automated synthetic scheme to yield the final form of the radiotracer.

In another embodiment of the invention, the chelator is conjugated to the HHRT ligand. An advantage of conjugating a chelator with a HHRT ligand is that the specific binding properties of the HHRT ligand can concentrate the radioactive signal over the area of interest. It is envisioned that the derivatives used for imaging and/or therapy may comprise a chelator conjugated to HHRT ligands designed for targeting cancerous tumors, pre-cancerous tumors, and/or disease functional pathways. The BFCA-HHRT ligand conjugate may also be used for assessing a pharmaceutical agent's effectiveness on various metabolic and/or biochemical pathways or individual reactions

It is contemplated that virtually any HHRT ligand that is known, or may be subsequently discovered may be used with the present invention. In certain embodiments, a HHRT ligand may be directly conjugated to a chelator (e.g., via a covalent bond between the targeting ligand and the chelator). Targeting ligands may be conjugated to different chelators, such as DTPA or DOTA and used for therapeutic purposes; in certain instances, it may be required to modify the HHRT ligand (e.g., adding a side chain that contains a hydroxyl or an amine) in order to covalently bind the targeting ligand to the different chelators.

The present invention further provides a method of synthesizing a radiolabeled BFCA-HHRT ligand conjugate for imaging or therapeutic use. For example, the method includes using the HHRT ligand SHH, admixing the said ligand with DOTA to obtain a DOTA-SHH conjugate, and admixing the said conjugate with a radionuclide to obtain a radiolabeled DOTA-SHH conjugate. The radionuclide is chelated to DOTA via an N₄ chelate. SHH is conjugated, as described above, to one acid arm of DOTA. As required, such as in the case of ^{99m}Tc and ^{186/188}Re, a reducing agent, preferably a dithionite ion, a stannous ion or a ferrous ion, is used for radiolabeling.

The present invention further provides a method for labeling a HHRT ligand for imaging, therapeutic, diagnostic or prognostic use. The labeling method includes the steps of obtaining a HHRT ligand, admixing the HHRT ligand with a BFCA to obtain a BFCA-HHRT ligand conjugate, and reacting the said conjugate with ⁶⁸Ga or ¹⁷⁷Lu to form coordination bond between the chelator and the ⁶⁸Ga or ¹⁷⁷Lu. For purposes of this embodiment, the HHRT ligand may be any of the ligands described above or discussed herein.

The present inventors have also discovered that it is possible to utilize a dual-conjugate approach by binding a second moiety (with or without specific targeting capabilities) to a component of the conjugated composition, such as a tissue targeting moiety, a therapeutic moiety, or an imaging moiety, such that the agent is suitable for multimodality targeting, imaging or radiochemotherapy.

Radioisotope Labeling

Generally, it is believed that virtually any α-emitter, β-emitter, γ-emitter, or β/γ-emitter can be used in conjunction with the invention. Exemplary α-emitters include ²¹¹At, ²¹²Bi and ²²³Ra. Preferred β-emitters include ⁹⁰Y and ²²⁵Ac. Exemplary β/γ-emitters include ⁶⁷Cu, ⁸⁹Sr, ¹⁵³Sm, ¹⁶⁶Ho, ¹⁷⁷Lu, ¹⁸⁶Re and ¹⁸⁸Re. Exemplary γ-emitters include ⁶²Cu, ⁶⁴Cu, ⁶⁷Ga, ⁶⁸Ga, ^{94m}Tc, ^{99m}Tc and ¹¹¹In. It is also envisioned that para-magnetic substances, such as Gd, Mn, Cu or Fe, can be chelated with DO2S derivatives for use in conjunction with the present invention.

In some aspects of radioimaging, the radiolabel is a gamma-radiation emitting radionuclide and the radiotracer is located using a gamma-radiation detecting camera (this process is often referred to as gamma scintigraphy). The imaged site is detectable because the radiotracer is chosen either to localize at a pathological site (termed positive contrast) or, alternatively, the radiotracer is chosen specifically not to localize at such pathological sites (termed negative contrast).

A variety of radioisotopes are known to be useful for radioimaging and radionuclide therapy, including ⁶⁷Ga, ⁶⁸Ga, ^{94m}Tc, ^{99m}Tc, ¹¹¹In, ¹²³I, ¹²⁵I, ¹⁶⁹Yb, ¹⁷⁷Lu, ¹⁸⁶Re and ¹⁸⁸Re, for example. Because of better imaging characteristics and cost-effectiveness, attempts have been made to replace or provide an alternative to ¹¹¹In-labeled compounds with corresponding ⁶⁸Ga labeled compounds when possible. Due to favorable physical characteristics as well as availability from a generator, ⁶⁸Ga is utilized for the labeling of diagnostic radiopharmaceuticals, in certain cases.

Numerous types of generator systems are known to those skilled in the art and any generator system that produces a sufficient quantity of a daughter nuclide can be useful in medical imaging including, but not limited to: ⁴⁴Ti/⁴⁴Sc, ⁵²Fe/^{52m}Mn, ⁶²Zn/⁶²Cu, ₆₈Ge/⁶⁸Ga, ⁷²Se/⁷²As, ⁸²Sr/⁸²Rb, ⁹⁹Mo/^{99m}Tc, ¹¹⁸Te/¹¹⁸Sb, ¹²²Xe/¹²²I, ¹²⁸Ba/¹²⁸Cs, ¹⁷⁸W/¹⁷⁸Ta, ¹⁸⁸W/¹⁸⁸Re, and ^{195m}Hg/^{195m}Au, for example.

A number of factors may be considered for optimal radioimaging in humans. In certain embodiments, a BFCA-HHRT ligand may be labeled (e.g., chelated) with ⁶⁸Ga for PET imaging or ¹⁷⁷Lu (a β and γ-emitter) for internal radionuclide therapy, for example. When chelated with non-radioactive metals (e.g. copper, cobalt, platinum, iron, arsenic, rhenium, germanium), the cold (non-radioactive) BFCA-HHRT ligand may be used as a metallic chemotherapeutic agent.

Therapeutic radionuclides emit radiation that interacts with tissues and cellular components typically resulting in cellular damage. Virtually any α-emitter, β-emitter, or auger electron-emitter can exert a therapeutic effect on its target. Pure β-emitters have longer pathlengths in tissue and are preferred for larger tumors; however, they lack imaging capabilities and utilize a diagnostic surrogate to provide biodistribution and dosimetry information. Certain radionuclides possess both β and γ-emissions allowing for a diagnostic scan of the agent using low radioactive doses, followed by increasing radioactive doses to treat the site of interest. ¹⁷⁷Lu is an example of a β/γ-emitting radionuclide that can be used with this invention to prepare a targeted agent with diagnostic and therapeutic characteristics. Other examples of β,/γ-emitters include ⁸⁹Sr, ¹⁵³Sm, ¹⁶⁶Ho, ¹⁸⁶Re and ¹⁸⁸Re. Due to favorable decay characteristics such as half-life (6.73 days), beta emission (490 keV), gamma emission (113 keV [6.4%], 208 keV [11%]) and feasible production route, ¹⁷⁷Lu is utilized for the labeling of therapeutic radionuclides, in certain cases.

### VI. Exemplary Kits of the Invention

The invention also provides a kit for preparing a radiopharmaceutical preparation and/or using the preparation in a therapeutic and/or diagnostic embodiment. In certain aspects, the kit includes one or more sealed vials or bags, or any other kind of appropriate container, containing a predetermined quantity of a chelator and HHRT ligand composition to label the conjugate with a radioisotope. The HHRT ligand may be any ligand that specifically binds to a hedgehog signaling tissue type, such as those discussed herein. In some cases, the kit comprises an additional cancer diagnostic or anti-cancer therapeutic agent, including chemotherapeutics, immunotherapies, radioisotopes, and so forth.

The components of the kit may be in any appropriate form, such as in liquid, frozen or dry form. In a preferred embodiment, the kit components are provided in lyophilized form. The kit may also include an antioxidant and/or a scavenger, in certain embodiments. The antioxidant may be any known antioxidant but is preferably vitamin C. Scavengers may also be present to bind unreacted radionuclide. Most commercially-available kits contain glucoheptonate as the scavenger. However, glucoheptonate does not completely react with typical kit components, leaving approximately 10-15% of unused material. This remaining glucoheptonate will go to a tumor and skew imaging results. Therefore, in certain embodiments DTPA, EDTA or DOTA is employed as the scavenger as they are cheaper and react more completely. Any components of the kit may be provided in separate containers or may be provided already put together.

Complexes and means for preparing such complexes may be provided in a kit form that typically includes a sealed vial containing a predetermined quantity of a chelator of the invention to label the chelator conjugate with a radionuclide. In some embodiments of the present invention, the kit includes a radionuclide. In certain further embodiments, the radionuclide is ⁶⁸Ga or ¹⁷⁷Lu, for example. The kit may also contain conventional pharmaceutical adjunct materials such as, for example, pharmaceutically acceptable salts to adjust the osmotic pressure, buffers, preservatives, antioxidants, and the like. Reducing agents may also be included in kits when the radioisotope is ^{99m}Tc or ¹⁸⁸Re, for example.

In certain embodiments, an antioxidant and a transition chelator are included in the composition to prevent oxidation of the chelator conjugate. In certain embodiments, the antioxidant is vitamin C (ascorbic acid). However, it is contemplated that any other antioxidant known to those of ordinary skill in the art, such as tocopherol, pyridoxine, thiamine, or rutin, may also be used. Examples of transition chelators for use in the present invention include, but are not limited to, glucoheptonate, gluconate, glucarate, citrate, and tartarate. The components of the kit may be in liquid, frozen or dry form. In certain embodiments, kit components may be provided in lyophilized form.

### VII. Uses for HHRT Ligand Conjugates

The HHRT ligand conjugates of the invention may be used for diagnosis. It is envisioned that HHRT ligand conjugates may be administered to a patient having a tumor and effectively localize in the tumor site through targeting the HH pathway. Baseline imaging studies may be performed to determine the presence of the HH receptors on the tumor and provide diagnostic information about the disease. Once the patient is given a prescribed course of therapy (i.e. chemotherapy, radiation therapy), follow-up diagnostic scans can be performed with radiolabeled HHRT ligand conjugates to evaluate the effect on HH receptor status and serve as a biomarker for treatment monitoring.

The present invention may also be used to monitor the progress of former patients who have undergone chemotherapy or radiation treatment to determine if cancer has remained in remission or is metastasizing. People with a history of cancer in their family or who have been diagnosed with a genotype(s) associated with cancer may undergo monitoring by health professionals using the methodology of the current invention. The methods and pharmaceutical agents of the current invention may also be used by a health professional to monitor if cancer has started to develop in a person with cancer risk factors, such as environmental exposure to carcinogens, for example. Such methods to monitor the progress and/or recurrence of cancer and other diseases, known to those of skill in the art, are all applicable to the present invention.

The present invention may also be used for the delivery of radionuclide therapy. A therapeutic radionuclide may be chelated by a BFCA-HHRT ligand conjugate and used for targeted treatment of disease. For example, ¹⁷⁷Lu has a beta emission of 498 keV, which is suitable for therapy, and it also possesses a gamma emission that can allow for accurate dosimetry and imaging of ¹⁷⁷Lu-labeled compounds. The ability to directly image and assess the biodistribution and dosimetry of therapeutic radionuclides in vivo will assist in determining target specificity as well as validating the localization of dose over time. Chelation of ¹⁷⁷Lu to a BFCA-HHRT ligand conjugate would allow targeting of the radionuclide complex to tumor cells and spare non-target organs from unnecessary radiation dose.

The present invention includes embodiments that are useful for the targeted delivery of metallic therapy. Toxic metals can be chelated to BFCA-HHRT ligand conjugates and used for the treatment of cancer. Metals of interest include but are not limited to gallium, iron, arsenic and platinum, for example. It is envisioned that such an approach would increase specificity of drug delivery with reduced systemic toxicity, which is typically associated with non-targeted delivery of such metals. A radiotracer using the radioactive form of the respective metal could serve as a guide for biodistribution, selection of response in different tumor types, and pharmacokinetic characterization. This and related embodiments of the present invention are known to those having skill in the art upon the disclosure of the present invention.

### VIII. Drug Assessment

Radiolabeled agents can be applied in measuring treatment assessment. Certain HHRT ligands of the present invention can be applied in measuring the pharmacological response of a subject to a drug or therapeutic regimen in what is known as “image-guided therapy”.

### IX. Combination Therapy

It is an aspect of this invention that BFCA-HHRT ligand conjugates, such as radiolabeled BFCA-HHRT ligand conjugates, can be used in combination with another agent or therapy method, such as another cancer treatment. The BFCA-HHRT ligand conjugate may precede or follow the other agent treatment by intervals ranging from minutes to weeks. In embodiments where the other agent and the composition of the invention are applied separately to the cell, one would generally ensure that a significant period of time did not expire between the time of each delivery, such that the agent and composition of the invention would still be able to exert an advantageously combined effect on the cell. For example, in such instances, it is contemplated that one may contact the cell, tissue or organism with one, two, three, four or more modalities substantially simultaneously (i.e., within less than about a minute) with the BFCA-HHRT ligand conjugate. In other aspects, one or more agents may be administered within about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, to about 48 hours or more prior to and/or after administering the BFCA and HHRT ligand composition. In certain other embodiments, an agent may be administered within of from about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20, to about 21 days prior to and/or after administering the BFCA and HHRT ligand composition, for example. In some situations, it may be desirable to extend the time period for treatment significantly, such as where several weeks (e.g., about 1, about 2, about 3, about 4, about 5, about 6, about 7 or about 8 weeks or more) lapse between the respective administrations.

Various combinations may be employed, the BFCA-HHRT ligand conjugate is “A” and the secondary agent, which can be any other cancer therapeutic agent, is

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity. It is expected that the treatment cycles would be repeated as necessary. It also is contemplated that various standard therapies, as well as surgical intervention, may be applied in combination with the BFCA-HHRT ligand. The additional therapies include but are not limited to chemotherapy, radiotherapy, immunotherapy, gene therapy and surgery, for example.

A. Chemotherapy

Cancer therapies also include a variety of combination therapies with both chemical and radiation based treatments. Combination chemotherapy includes, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein tansferase inhibitors, COX-2 inhibitors, cholesterol synthesis inhibitors, cisplatinum, 5-fluorouracil, vincristin, vinblastin, staurosporine, streptozocin, fludurabine, methotrexate, genistein, curcumin, resveratrol, silymarin, caffeic acid phenethyl ester, flavopiridol, emodin, green tea polyphenols, piperine, oleandrin, ursolic acid, butamic acid, actinomycin D, thalidomide or any analog or derivative variant of the foregoing.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Radiochemotherapy

Radiochemotherapy is the combined delivery of radiation and chemotherapy to a target. This can be achieved in a single agent through conjugation of a chemotherapeutic agent to a BFCA-HHRT ligand conjugate, which is then subsequently radiolabeled with a therapeutic radionuclide. Combinations of radiochemotherapy include, for example, cisplatin (CDDP) with α-emitters, cyclophosphamide with β-emitters, doxorubicin with β/γ-emitters and taxol with Auger-emitters, or any analog or derivative variant of the foregoing.

D. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionucleotide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy could thus be used as part of a combined therapy, possibly in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155, for example.

E. Gene Therapy

In yet another embodiment, the secondary treatment is a gene therapy in which a therapeutic polynucleotide is administered before, after, or at the same time a first therapeutic agent. Delivery of the therapeutic agent in conjunction with a vector encoding a gene product will have a combined anti-hyperproliferative effect on target tissues, in certain cases.

F. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. Curative surgery includes resection in which all or part of cancerous tissue is physically or partially removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

### X. Pharmaceutical Compositions

Pharmaceutical compositions of the present invention comprise an effective amount of a composition of the invention, for example a BFCAand HHRT ligand conjugate of the present invention, dissolved or dispersed in a pharmaceutically acceptable carrier. The phrases “pharmaceutical” or “pharmacologically acceptable” refer to molecular entities and compositions that do not produce an adverse, allergic or other untoward reaction when administered to an animal, such as, for example, a human, as appropriate. The preparation of a pharmaceutical composition that contains at least one BFCA-HHRT ligand, such as a radiolabeled BFCA-HHRT ligand conjugate, and in some cases an additional active ingredient, will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The BFCA-HHTR ligand conjugates of the present invention may comprise different types of carriers depending on whether it is to be administered in solid, liquid or aerosol form, and whether it needs to be sterile for such routes of administration such as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a BFCA-HHRT ligand. In other embodiments, the active compound may comprise between about 2% to about 75% of the weight of the unit, or between about 25% to about 60%, for example, and any range derivable therein. In other non-limiting examples, a dose may also comprise from about 0.1 mg/kg/body weight, 0.5 mg/kg/body weight, 1 mg/kg/body weight, about 5 mg/kg/body weight, about 10 mg/kg/body weight, about 20 mg/kg/body weight, about 30 mg/kg/body weight, about 40 mg/kg/body weight, about 50 mg/kg/body weight, about 75 mg/kg/body weight, about 100 mg/kg/body weight, about 200 mg/kg/body weight, about 350 mg/kg/body weight, about 500 mg/kg/body weight, about 750 mg/kg/body weight, to about 1000 mg/kg/body weight or more per administration, and any range derivable therein. In non-limiting examples of a derivable range from the numbers listed herein, a range of about 10 mg/kg/body weight to about 100 mg/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

The BFCA-HHRT ligand conjugate may be formulated into a composition in a free base, neutral or salt form. Pharmaceutically acceptable salts include the salts formed with the free carboxyl groups of certain BFCAs (i.e. DO2S) derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example, liquid polyol or lipids; by the use of surfactants such as, for example, hydroxypropylcellulose; or combinations thereof such methods. In many cases, it will be preferable to include isotonic agents, such as, for example, sugars, sodium chloride or combinations thereof.

Sterile injectable solutions are prepared by incorporating the instant invention in the required amount of the appropriate solvent with various amounts of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### XI. Imaging

Functional imaging modalities (for example, positron emission tomography, PET; single photon emission computed tomography, SPECT) use radiotracers to image, map and measure biological attributes of tumors, such as metabolism, proliferation and surface receptor expression

Certain embodiments of the present invention provide a method of imaging a site within a mammalian body. For example, the imaging method includes the steps of administering an effective diagnostic amount of a composition comprising a ⁶⁸Ga labeled BFCA-HHRT ligand conjugate and detecting the radioactive signal from the ⁶⁸Ga localized at the site. The detecting step will typically be performed from about 10 minutes to about 4 hours after introduction of the composition into the mammalian body. Most preferably, the detecting step will be performed about 1 hour after injection of the ⁶⁸Ga composition into the mammalian body.

The HHRT ligand conjugate may also be used as a diagnostic tool and/or for predicting responses to certain kinds of treatment. For example, DTPA-SHH can be labeled with the gamma-emmiting isotopes ^{99m}Tc and may be used to image cancerous tumors; in this example, the imaging may provide important information about the disease such as: 1) to what degree the cancerous cells express the PTCH receptor and 2) how can the receptor expression characterization be used to predict how the disease will respond to HH receptor-targeted therapy (e.g., when it is identified that cancerous tumors selectively express high levels of hedgehog receptor, this information indicates that the cancerous cells will likely respond to therapeutic doses of anti-cancer agents that target cells expressing the hedgehog receptor). This approach is referred to as “image guided therapy”.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of ⁶⁸Ga-DOTA-SHH and Determination of Stability

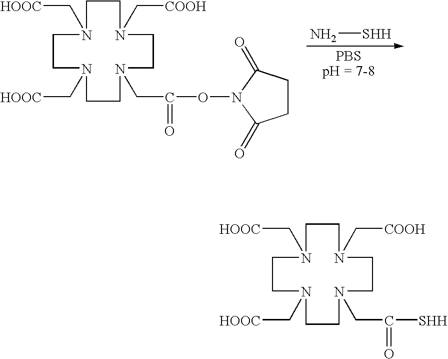

DOTA-SHH was prepared by coupling of DOTA-NHS to the 19.5 kDa human N-terminal SHH protein (R&D Systems). DOTA-NHS (1.25 μmol) in 2 mL of phosphate buffer (pH=7.5), 0.15 mL of 0.1 M DTT and 0.4 mL of 0.2 M imidazole (pH=8) were added to solution of the SSH (0.025 μmol) in 1×PBS buffer while on ice. The reaction was carried out at 4° C. for 20 hrs. The product was purified and concentrated by ultracentrifugation through the Amicon filter to remove hydrolyzed DOTA. DOTA-SHH was obtained in 45%-60% total yield assessed by RP-HPLC. DOTA-SHH was characterized using MALDI-TOF with purity >90%. The resulting conjugate was labeled with ⁶⁸Ga in acetate buffer (pH=4) and heated at 37° C. for 30 min. Radiochemical purity was >97% and HPLC analysis showed the complex was unchanged throughout the labeling reaction and no degradation products were observed.

The stability of ⁶⁸Ga-SHH in serum was determined. ⁶⁸Ga-SHH was labeled as previously described. The radiolabeled agent was transferred into an eppendorf tube containing 1 ml of FBS. The sample was incubated at 37° C. and aliquots were removed and assayed via radio-instant thin layer chromatography (ITLC) at 10, 30, 60 and 90 mins post-incubation. 4 mM EDTA (pH 4) was used as the mobile phase. Serum stability data of ⁶⁸Ga-SHH are shown in FIG. 3. The data show no significant decrease in stability of the radiolabeled complex over the course of the study. This is expected as the ⁶⁸Ga-binding core exhibits favorable coordination of radiometals under physiologic challenge

### Example 2

### The In Vitro Biokinetics of ⁶⁸GA-DOTA-SHH in Cancer Cell Lines with Active HH Signaling Pathways

To ensure that the chemical modifications to SHH peptide did not alter its ability to bind to the PTCH receptor, the in vitro bioactivity of ⁶⁸Ga-DOTA-SHH was investigated.

In vitro bioactivity of ⁶⁸Ga-DOTA-SHH was evaluated using binding studies in the HH receptor positive breast cancer cell lines BT-474 and MDA-MB-231 and prostate cancer cell lines DU145 and RV221. Cells were seeded at a density of 2*10⁵ in 6 well plates and grown overnight. Cells were incubated with 1-2 μCi of ⁶⁸Ga-DOTA-SHH for 15-120 min. At the end of each time point, the radioactivity in the cells and media were collected and counted. The percent uptake was calculated as the ratio of cpm (cells)/cpm (media). Receptor saturation was observed between 120 and 240 min. The amount of receptor binding of ⁶⁸Ga-DOTA-SHH correlates with PTCH receptor expression on each cell line.

### Example 3

### Radiolabeling of DOTA-SHH with Lu-177

Thirty micrograms of DOTA-SHH (synthesized in example 1) was dissolved in 0.2 M sodium acetate buffer containing ascorbic acid (pH ˜5.5). Ten mCi of Lu-177 chloride was added to the solution and heated at 37° C. for 1 hour. The product was purified by HPLC and showed >98% radiochemical purity.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

### PATENTS

U.S. Pat. No. 4,141,654
U.S. Pat. No. 5,605,672
U.S. Pat. No. 5,648,063
U.S. Pat. No. 5,880,281
U.S. Pat. No. 6,071,490
U.S. Pat. No. 6,613,305
U.S. Pat. No. 6,737,247

### PUBLICATIONS

Agouni, A., H. A. Mostefai, et al. (2007). “Sonic hedgehog carried by microparticles corrects endothelial injury through nitric oxide release.” Faseb J 21(11): 2735-41.
Anton Aparicio, L. M., R. Garcia Campelo, et al. (2007). “Prostate cancer and Hedgehog signaling pathway.” Clin Transl Oncol 9(7): 420-8.
Asai, J., H. Takenaka, et al. (2006). “Topical sonic hedgehog gene therapy accelerates wound healing in diabetes by enhancing endothelial progenitor cell-mediated microvascular remodeling.” Circulation 113(20): 2413-24.
Bakheet, S. M. & Powe, J. Benign causes of 18-FDG uptake on whole body imaging. Semin Nucl Med 28, 352-358 (1998).
Bale, A. E. and K. P. Yu (2001). “The hedgehog pathway and basal cell carcinomas.” Hum Mol Genet 10(7): 757-62.
Bar, E. E., A. Chaudhry, et al. (2007). “Cyclopamine-Mediated Hedgehog Pathway Inhibition Depletes Stem-Like Cancer Cells in Glioblastoma.” Stem Cells 25(10): 2524-2533.
Berman, D. M., S. S. Karhadkar, et al. (2003). “Widespread requirement for Hedgehog ligand stimulation in growth of digestive tract tumours.” Nature 425(6960): 846-51.
Berman, D. M. et al. Widespread requirement for Hedgehog ligand stimulation in growth of digestive tract tumours. Nature 425, 846-851 (2003).
Brenner, B., Ilson, D. H. & Minsky, B. D. Treatment of localized esophageal cancer. Semin Oncol 31, 554-565 (2004).
Bumcrot D A, Takada R and McMahon AP (1995). “Proteolytic processing yields two secreted forms of sonic hedgehog”. Mol Cell Biol. 15 (4): 2294-2303.
Chen, J. K., J. Taipale, et al. (2002). “Inhibition of Hedgehog signaling by direct binding of cyclopamine to Smoothened.” Genes Dev 16(21): 2743-8.
Couve-Privat, S., M. Le Bret, et al. (2004). “Functional analysis of novel sonic hedgehog gene mutations identified in basal cell carcinomas from xeroderma pigmentosum patients.” Cancer Res 64(10): 3559-65.
Cutcliffe, C., D. Kersey, et al. (2005). “Clear cell sarcoma of the kidney: up-regulation of neural markers with activation of the sonic hedgehog and Akt pathways.” Clin Cancer Res 11(22): 7986-94.
Dai, P., H. Akimaru, et al. (1999). “Sonic Hedgehog-induced activation of the Gli1 promoter is mediated by GLI3.” J Biol Chem 274(12): 8143-52.
Dean, M., Fojo, T. & Bates, S. Tumour stem cells and drug resistance. Nat Rev Cancer 5, 275-284 (2005).
Dimmler, A., T. Brabletz, et al. (2003). “Transcription of sonic hedgehog, a potential factor for gastric morphogenesis and gastric mucosa maintenance, is up-regulated in acidic conditions.” Lab Invest 83(12): 1829-37.
Downey, R. J. et al. Whole body 18FDG-PET and the response of esophageal cancer to induction therapy: results of a prospective trial. J Clin Oncol 21, 428-432 (2003).
Ehtesham, M., A. Sarangi, et al. (2007). “Ligand-dependent activation of the hedgehog pathway in glioma progenitor cells.” Oncogene 26(39): 5752-61.
Ericson, J., S. Morton, et al. (1996). “Two critical periods of Sonic Hedgehog signaling required for the specification of motor neuron identity.” Cell 87(4): 661-73.
Flamen, P. et al. Positron emission tomography for assessment of the response to induction radiochemotherapy in locally advanced oesophageal cancer. Ann Oncol 13, 361-368 (2002).
Flamen, P. et al. Utility of positron emission tomography for the staging of patients with potentially operable esophageal carcinoma. J Clin Oncol 18, 3202-3210 (2000).
Frank-Kamenetsky, M., X. M. Zhang, et al. (2002). “Small-molecule modulators of Hedgehog signaling: identification and characterization of Smoothened agonists and antagonists.” J Biol 1(2): 10.
Fukaya, M., N. Isohata, et al. (2006). “Hedgehog signal activation in gastric pit cell and in diffuse-type gastric cancer.” Gastroenterology 131(1): 14-29.
Harmon, E. B., A. H. Ko, et al. (2002). “Hedgehog signaling in gastrointestinal development and disease.” Curr Mol Med 2(1): 67-82.
Howell, R. W. et al. The MIRD perspective 1999. Medical Internal Radiation Dose Committee. J Nucl Med 40, 3S-10S (1999).
Ingham, P. W. and A. P. McMahon (2001). “Hedgehog signaling in animal development: paradigms and principles.” Genes Dev 15(23): 3059-87.
Iyer, R., Wilkinson, N., Demmy, T. & Javle, M. Controversies in the multimodality management of locally advanced esophageal cancer: evidence-based review of surgery alone and combined-modality therapy. Ann Surg Oncol 11, 665-673 (2004).
Jones, D. R., Parker, L. A., Jr., Detterbeck, F. C. & Egan, T. M. Inadequacy of computed tomography in assessing patients with esophageal carcinoma after induction chemoradiotherapy. Cancer 85, 1026-1032 (1999).
Kayed, H., J. Kleeff, et al. (2005). “Localization of the human hedgehog-interacting protein (Hip) in the normal and diseased pancreas.” Mol Carcinog 42(4): 183-92.
Knoess, C. et al. Performance evaluation of the microPET R4 PET scanner for rodents. Eur J Nucl Med Mol Imaging 30, 737-747 (2003).
Larson, S. M. Cancer or inflammation? A Holy Grail for nuclear medicine. J Nucl Med 35, 1653-1655 (1994).
Lauth, M., A. Bergstrom, et al. (2007). “Inhibition of GLI-mediated transcription and tumor cell growth by small-molecule antagonists.” Proc Natl Acad Sci USA 104(20): 8455-60.
Law, S., Fok, M., Chow, S., Chu, K. M. & Wong, J. Preoperative chemotherapy versus surgical therapy alone for squamous cell carcinoma of the esophagus: a prospective randomized trial. J Thorac Cardiovasc Surg 114, 210-217 (1997).
Lee, J., X. Wu, et al. (2007). “A Small-Molecule Antagonist of the Hedgehog Signaling Pathway.” Chembiochem.

Lee, S. Y., H. S. Han, et al. (2007). “Sonic hedgehog expression in gastric cancer and gastric adenoma.” Oncol Rep 17(5): 1051-5.
Levanat, S., V. Musani, et al. (2004). “Role of the hedgehog/patched signaling pathway in oncogenesis: a new polymorphism in the PTCH gene in ovarian fibroma.” Ann N Y Acad Sci 1030: 134-43.
Liu, M. S., P. Y. Yang, et al. (2007). “Sonic hedgehog signaling pathway in pancreatic cystic neoplasms and ductal adenocarcinoma.” Pancreas 34(3): 340-6.
Luketich, J. D. et al. Evaluation of distant metastases in esophageal cancer: 100 consecutive positron emission tomography scans. Ann Thorac Surg 68, 1133-1136; discussion 1136-1137 (1999).
Ma, X. et al. Hedgehog signaling is activated in subsets of esophageal cancers. Int J Cancer (2005).
Ma, X. et al. Hedgehog signaling is activated in subsets of esophageal cancers. Int J Cancer 118, 139-148 (2006).
Ma, X., K. Chen, et al. (2005). “Frequent activation of the hedgehog pathway in advanced gastric adenocarcinomas.” Carcinogenesis 26(10): 1698-705.
Ma, X., T. Sheng, et al. (2005). “Hedgehog signaling is activated in subsets of esophageal cancers.” Int J. Cancer.
Ma, X., T. Sheng, et al. (2006). “Hedgehog signaling is activated in subsets of esophageal cancers.” Int J Cancer 118(1): 139-48.
Maecke, H. R., Hofmann, M. & Haberkorn, U. (68)Ga-labeled peptides in tumor imaging. J Nucl Med 46 Suppl 1, 172S-178S (2005).
Morton, J. P., M. E. Mongeau, et al. (2007). “Sonic hedgehog acts at multiple stages during pancreatic tumorigenesis.” Proc Natl Acad Sci USA 104(12): 5103-8.
Mukherjee, S., N. Frolova, et al. (2006). “Hedgehog signaling and response to cyclopamine differ in epithelial and stromal cells in benign breast and breast cancer.” Cancer Biol Ther 5(6): 674-83.
Nielsen, C. M., J. Williams, et al. (2004). “Hh pathway expression in human gut tissues and in inflammatory gut diseases.” Lab Invest 84(12): 1631-42.
Noveen, A., T. X. Jiang, et al. (1996). “cAMP, an activator of protein kinase A, suppresses the expression of sonic hedgehog.” Biochem Biophys Res Commun 219(1): 180-5.
Osipo, C. and L. Miele (2006). “Hedgehog signaling in hepatocellular carcinoma: novel therapeutic strategy targeting hedgehog signaling in HCC.” Cancer Biol Ther 5(2): 238-9.
Park, H. R. and Y. K. Park (2007). “Differential expression of runx2 and Indian hedgehog in cartilaginous tumors.” Pathol Oncol Res 13(1): 32-7.
Patil, M. A., J. Zhang, et al. (2006). “Hedgehog signaling in human hepatocellular carcinoma.” Cancer Biol Ther 5(1): 111-7.
Peacock, C. D., Q. Wang, et al. (2007). “Hedgehog signaling maintains a tumor stem cell compartment in multiple myeloma.” Proc Natl Acad Sci USA 104(10): 4048-53.
Pepinsky R B, Zeng C, Wen D, Rayhorn P, Baker D P, Williams K P, Bixler S A, Ambrose C M, Garber E A, Miatkowski K et al (1998). “Identification of a palmitic acid-modified form of human Sonic hedgehog”. J Biol Chem 273 (22): 14037-14045.
Porter J A, Young K E and Beachy P A (1996). “Cholesterol modification of hedgehog signaling proteins in animal development”. Science 274 (5285): 255-259.
Rao, G., C. A. Pedone, et al. (2004). “Sonic hedgehog and insulin-like growth factor signaling synergize to induce medulloblastoma formation from nestin-expressing neural progenitors in mice.” Oncogene 23(36): 6156-62.
Ruel, L., R. Rodriguez, et al. (2003). “Stability and association of Smoothened, Costal2 and Fused with Cubitus interruptus are regulated by Hedgehog.” Nat Cell Biol 5(10): 907-13.
Ruel, L., Rodriguez, R., Gallet, A., Lavenant-Staccini, L. & Therond, P. P. Stability and association of Smoothened, Costal2 and Fused with Cubitus interruptus are regulated by Hedgehog. Nat Cell Biol 5, 907-913 (2003).
Sengupta, A., D. Banerjee, et al. (2007). “Deregulation and cross talk among Sonic hedgehog, Wnt, Hox and Notch signaling in chronic myeloid leukemia progression.” Leukemia 21(5): 949-55.
Shafaee, Z., H. Schmidt, et al. (2006). “Cyclopamine increases the cytotoxic effects of paclitaxel and radiation but not cisplatin and gemcitabine in Hedgehog expressing pancreatic cancer cells.” Cancer Chemother Pharmacol 58(6): 765-70.
Shafaee, Z., Schmidt, H., Du, W., Posner, M. & Weichselbaum, R. Cyclopamine increases the cytotoxic effects of paclitaxel and radiation but not cisplatin and gemcitabine in Hedgehog expressing pancreatic cancer cells. Cancer chemotherapy and pharmacology 58, 765-770 (2006).
Sheng, T., C. Li, et al. (2004). “Activation of the hedgehog pathway in advanced prostate cancer.” Mol Cancer 3: 29.
Sicklick, J. K., Y. X. Li, et al. (2006). “Dysregulation of the Hedgehog pathway in human hepatocarcinogenesis.” Carcinogenesis 27(4): 748-57.
Sims-Mourtada, J., Izzo, J. G., Ajani, J. & Chao, K. S. Sonic Hedgehog promotes multiple drugresistance by regulation of drug transport. Oncogene (2007).
Sims-Mourtada, J., J. G. Izzo, et al. (2006). “Hedgehog: an attribute to tumor regrowth after chemoradiotherapy and a target to improve radiation response.” Clin Cancer Res 12(21): 6565-72.
Sims-Mourtada, J., J. G. Izzo, et al. (2007). “Sonic Hedgehog promotes multiple drug resistance by regulation of drug transport.” Oncogene.
Snijders, A. M., B. L. Schmidt, et al. (2005). “Rare amplicons implicate frequent deregulation of cell fate specification pathways in oral squamous cell carcinoma.” Oncogene 24(26): 4232-42.
Stabin, M. G. MIRDOSE: personal computer software for internal dose assessment in nuclear medicine. J Nucl Med 37, 538-546 (1996).
Stabin, M. G., Sparks, R. B. & Crowe, E. OLINDA/EXM: the second-generation personal computer software for internal dose assessment in nuclear medicine. J Nucl Med 46, 1023-1027 (2005).
Steg, A., W. Wang, et al. (2006). “Multiple gene expression analyses in paraffin-embedded tissues by TaqMan low-density array: Application to hedgehog and Wnt pathway analysis in ovarian endometrioid adenocarcinoma.” J Mol Diagn 8(1): 76-83.
Sui, G., P. Bonde, et al. (2006). “Epidermal growth factor receptor and hedgehog signaling pathways are active in esophageal cancer cells from rat reflux model.” J Surg Res 134(1): 1-9.
Suwelack, D., A. Hurtado-Lorenzo, et al. (2004). “Neuronal expression of the transcription factor Gli1 using the Talpha 1 alpha-tubulin promoter is neuroprotective in an experimental model of Parkinson's disease.” Gene Ther 11(24): 1742-52.
Swisher, S. G. et al. 2-Fluoro-2-deoxy-D-glucose positron emission tomography imaging is predictive of pathologic response and survival after preoperative chemoradiation in patients with esophageal carcinoma. Cancer 101, 1776-1785 (2004).
Thayer, S. P., M. P. di Magliano, et al. (2003). “Hedgehog is an early and late mediator of pancreatic cancer tumorigenesis.” Nature 425(6960): 851-6.
Thayer, S. P. et al. Hedgehog is an early and late mediator of pancreatic cancer tumorigenesis. Nature 425, 851-856 (2003).
Thievessen, I., M. Wolter, et al. (2005). “Hedgehog signaling in normal urothelial cells and in urothelial carcinoma cell lines.” J Cell Physiol 203(2): 372-7.
Tones, E. M., C. Monville, et al. (2005). “Delivery of sonic hedgehog or glial derived neurotrophic factor to dopamine-rich grafts in a rat model of Parkinson's disease using adenoviral vectors Increased yield of dopamine cells is dependent on embryonic donor age.” Brain Res Bull 68(1-2): 31-41.
Tuncer, M. C., H. Ozturk, et al. (2007). “Interaction of L-arginine-methyl ester and Sonic hedgehog in liver ischemia-reperfusion injury in the rats.” World J Gastroenterol 13(28): 3841-6.
Urba, S. Esophageal cancer: preoperative or definitive chemoradiation. Ann Oncol 15 Suppl 4, iv93-96 (2004).
Vestergaard, J., M. W. Pedersen, et al. (2006). “Hedgehog signaling in small-cell lung cancer: frequent in vivo but a rare event in vitro.” Lung Cancer 52(3): 281-90.
Vila, G., M. Theodoropoulou, et al. (2005). “Expression and function of sonic hedgehog pathway components in pituitary adenomas: evidence for a direct role in hormone secretion and cell proliferation.” J Clin Endocrinol Metab 90(12): 6687-94.
Watkins, D. N. and C. D. Peacock (2004). “Hedgehog signalling in foregut malignancy.” Biochem Pharmacol 68(6): 1055-60.
Watkins, D. N., D. M. Berman, et al. (2003). “Hedgehog signalling within airway epithelial progenitors and in small-cell lung cancer.” Nature 422(6929): 313-7.
Watkins, D. N. & Peacock, C. D. Hedgehog signalling in foregut malignancy. Biochem Pharmacol 68, 1055-1060 (2004).
Xuan, Y. H., H. S. Jung, et al. (2006). “Enhanced expression of hedgehog signaling molecules in squamous cell carcinoma of uterine cervix and its precursor lesions.” Mod Pathol 19(8): 1139-47.
Yoon, J. W., Y. Kita, et al. (2002). “Gene expression profiling leads to identification of GLI1-binding elements in target genes and a role for multiple downstream pathways in GLI1-induced cell transformation.” J Biol Chem 277(7): 5548-55.

Yoon, J. W. et al. Gene expression profiling leads to identification of GLI1-binding elements in target genes and a role for multiple downstream pathways in GLI1-induced cell transformation. J Biol Chem 277, 5548-5555 (2002).
Zuccaro, G., Jr. et al. Endoscopic ultrasound cannot determine suitability for esophagectomy after aggressive chemoradiotherapy for esophageal cancer. Am J Gastroenterol 94, 906-912 (1999).

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A composition comprising:
a hedgehog receptor targeting ligand;
a chelator, said chelator conjugated to said ligand; and
a metal.

2. The composition of claim 1, wherein the hedgehog receptor targeting ligand is hedgehog, or a fragment thereof that binds to the hedgehog receptor.

3. The composition of claim 2, wherein the hedgehog fragment is further defined as a polypeptide of 10 or more amino acids comprising at least 70% identity, at least 75% identity, at least 80% identity, at least 85% identity, at least 90% identity, at least 95% identity, at least 97% identity, at least 98% identity, or at least 99% identity to SEQ ID NO:1.

4. The composition of claim 1, wherein the chelator is a chelating group comprised of N, O and/or S atoms.

5. The composition of claim 4, wherein the chelating group is selected from the group consisting of ethylenediaminetetraacetic acid; diethylenetriaminepentaacetic acid (DTPA); 1,4,7,10-tetraazacyclododecane-N,N′,N″,N′″-tetraacetic acid (DOTA); 1,4,8,11-tetraazacyclotetradecane-N,N′,N″,N′″-tetraacetic acid (TETA); 1,4,8,12-tetraazacyclopentadecane-N,N′,N″,N′″-tetraacetic acid (15N4); 1,4,7-triazacyclononane-N,N′,N″-triacetic acid (9N3); 1,5,9-triazacyclododecane-N,N′,N″-triacetic acid (12N3); 2-p-nitrobenzyl-1,4,7,10-tetraazacyclododecane-N,N′,N″,N′″-tetraacetic acid; and 6-bromoacetamido-benzyl-1,4,8,11-tetraazacyclotetadecane-N,N′,N″,N′″-tetraacetic acid (BAT).

6. The composition of claim 1, wherein the metal species is a radionuclide.

7. The composition of claim 6 wherein the radionuclide is Ti, Fe, Cu, ⁶¹Cu, ⁶²Cu, ⁶⁴Cu, ⁶⁷Cu, ⁶⁷Ga, ⁶⁸Ga, ⁸⁹Sr, ⁹⁰Y, ^{94m}Tc, ^{99m}Tc, ¹¹¹In, ¹⁴⁹Pm, ¹⁵³Gd, ¹⁵³Sm, ¹⁶⁶Ho, ¹⁷⁷Lu, ¹⁸⁶Re, ¹⁸⁸Re, ²¹¹At, ²¹²Bi, or ²²⁵Ac.

8. The composition of claim 1, wherein said metal species is copper, cobalt, platinum, iron, arsenic, rhenium, or germanium.

9. The composition of claim 1, wherein the metal is a paramagnetic ion.

10. The composition of claim 1, wherein the composition further comprises a pharmaceutically acceptable excipient.

11. A method for the diagnosis or treatment of a medical condition in a subject comprising:
administering to the subject a composition of:
a hedgehog receptor targeting ligand,
a chelator, said chelator conjugated to said ligand, and
a metal;
and
imaging said subject and/or treating said subject.

12. The method of claim 11, wherein the hedgehog receptor targeting ligand is hedgehog, or a fragment thereof that binds to the hedgehog receptor.

13. The method of claim 12, wherein the hedgehog fragment is further defined as a polypeptide of 10 or more amino acids comprising at least 70% identity, at least 75% identity, at least 80% identity, at least 85% identity, at least 90% identity, at least 95% identity, at least 97% identity, at least 98% identity, or at least 99% identity to SEQ ID NO:1.

14. The method of claim 11, wherein the chelator is a chelating group comprised of N, O and/or S atoms.

15. The method of claim 14, wherein the chelating group is selected from the group consisting of ethylenediaminetetraacetic acid; diethylenetriaminepentaacetic acid (DTPA); 1,4,7,10-tetraazacyclododecane-N,N′,N″,N′″-tetraacetic acid (DOTA); 1,4,8,11-tetraazacyclotetradecane-N,N′,N″,N′″-tetraacetic acid (TETA); 1,4,8,12-tetraazacyclopentadecane-N,N′,N′,N′″-tetraacetic acid (15N4); 1,4,7-triazacyclononane-N,N′,N″-triacetic acid (9N3); 1,5,9-triazacyclododecane-N,N′,N″-triacetic acid (12N3); 2-p-nitrobenzyl-1,4,7,10-tetraazacyclododecane-N,N′,N″,N′″-tetraacetic acid; and 6-bromoacetamido-benzyl-1,4,8,11-tetraazacyclotetadecane-N,N′,N″,N′″-tetraacetic acid (BAT).

16. The method of claim 11, wherein said metal species is a radionuclide.

17. The method of claim 16 wherein said radionuclide is ⁴⁵Ti, ⁵⁹Fe, ⁶⁰Cu, ⁶¹Cu, ⁶²Cu, ⁶⁴Cu, ⁶⁷Ga, ⁶⁷Ga, ⁸⁹Sr, ⁹⁰Y, ^{94m}TC, ^{99m}Tc, ¹¹¹In, ¹⁴⁹Pm, ¹⁵³Gd, ¹⁵³Sm, ¹⁶⁶Ho, ¹⁷⁷Lu, ¹⁸⁶Re, ¹⁸⁸Re, ²¹¹At, ²¹²Bi, or ²²⁵Ac.

18. The method of claim 11, wherein said metal species is copper, cobalt, platinum, iron, arsenic, rhenium, or germanium.

19-22. (canceled)

23. The method of claim 11, wherein the medical condition is cancer.

24. The method of claim 23, wherein the cancer is basal cell carcinoma, medulloblastoma, hepatocellular carcinoma, pituitary carcinoma, glioblastoma, skin cancer, gall bladder cancer, spleen cancer, cartilaginous tumors, breast cancer, prostate cancer, uterine cancer, cervical cancer, ovarian cancer, small cell lung cancer, urothelial carcinoma, gastric cancer, esophageal cancer, pancreatic cancer, kidney cancer, neural tumors, liver cancer, testicular cancer and/or multiple myeloma.

25-46. (canceled)


SEQUENCE LISTING (3 sequences)

SEQ ID NO: 1 - protein, 173 aa, Homo sapiens
CGPGRGFGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSG

SEQ ID NO: 2 - protein, 395 aa, Homo Sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPRLLHALGALLPGGAVQPTGMHWYSRLLYRLAEELLG

SEQ ID NO: 3 - protein, 411 aa, Homo Sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS